(19) United States
(12) Patent Application Publication
(10) Pub. No.: US 2019/0054209 A1
(43) Pub. Date: Feb. 21, 2019

(54) ARTIFICAL CORNEA STROMA MATERIAL, AND ARTIFICAL CORNEA STROMA AND ARTIFICAL CORNEA SCAFFOLD OF SUCH MATERIAL

(71) Applicant: HON HAI PRECISION INDUSTRY CO., LTD., New Taipei (TW)

(72) Inventor: HSIU-WEN CHIEN, New Taipei (TW)

(21) Appl. No.: 15/680,339

(22) Filed: Aug. 18, 2017

(30) Foreign Application Priority Data
Aug. 15, 2017 (CN) 201710696706.X

(51) Int. Cl. A61L 27/26; A61L 27/50; A61F 2/14

(52) U.S. Cl. CPC A61L 27/26; A61L 2430/16; A61F 2/142; A61L 27/50

(57) ABSTRACT

An artificial cornea stroma material comprises a methacrylated hyaluronic acid, a collagen, a photo-initiator, and a solvent. Ultraviolet radiation of the photo-initiator causes the methacrylated hyaluronic acid to polymerize to form a dense covalent bond cross-linking network gel structure. The gel structure has mechanical strength, and can keep in gel state for a long time. An artificial cornea stroma made from the artificial material, and an artificial cornea scaffold using the artificial cornea stroma are also provided.

### FIELD

The subject matter herein generally relates to an artificial cornea stroma material, an artificial cornea stroma making by the artificial cornea stroma material, and an artificial cornea scaffold using the artificial cornea stroma.

### BACKGROUND

Cornea is an optically transparent tissue without blood vessels. Cornea can refract and filter light getting into the eyes. Cornea comprises an epithelial layer, a Bowman's layer, a stroma layer, a Descemet's membrane, and an endothelium. The Bowman's layer, the stroma layer, and the Descemet's membrane form a natural cornea scaffold. The natural cornea scaffold is non-renewable, if the cornea scaffold is damaged, the cornea is damaged. Therefore, an artificial cornea scaffold is needed.

### BRIEF DESCRIPTION OF THE DRAWINGS

Implementations of the present technology will now be described, by way of example only, with reference to the attached figures.

FIG. 1 is a diagrammatic view of an exemplary embodiment of an artificial cornea stroma.

FIG. 2 is a flow chart of an exemplary embodiment of a method for manufacturing the artificial cornea stroma of FIG. 1.

FIG. 3 is a diagrammatic view of an exemplary embodiment of an artificial cornea scaffold.

FIG. 4 is a flow chart of an exemplary embodiment of a method for manufacturing the artificial cornea scaffold of FIG. 3.

### DETAILED DESCRIPTION

It will be appreciated that for simplicity and clarity of illustration, where appropriate, reference numerals have been repeated among the different figures to indicate corresponding or analogous elements. In addition, numerous specific details are set forth in order to provide a thorough understanding of the embodiments described herein. However, it will be understood by those of ordinary skill in the art that the embodiments described herein can be practiced without these specific details. In other instances, methods, procedures, and components have not been described in detail so as not to obscure the related relevant feature being described. Also, the description is not to be considered as limiting the scope of the embodiments described herein. The drawings are not necessarily to scale, and the proportions of certain parts may be exaggerated to illustrate details and features of the present disclosure better. The disclosure is illustrated by way of example and not by way of limitation in the figures of the accompanying drawings, in which like references indicate similar elements. It should be noted that references to “an” or “one” embodiment in this disclosure are not necessarily to the same embodiment, and such references mean “at least one.”

The term “comprising” when utilized, means “including, but not necessarily limited to”; it specifically indicates open-ended inclusion or membership in the so-described combination, group, series, and the like. The term “about” when utilized, means “not only includes the numerical value, but also includes numbers closest to the numerical value”.

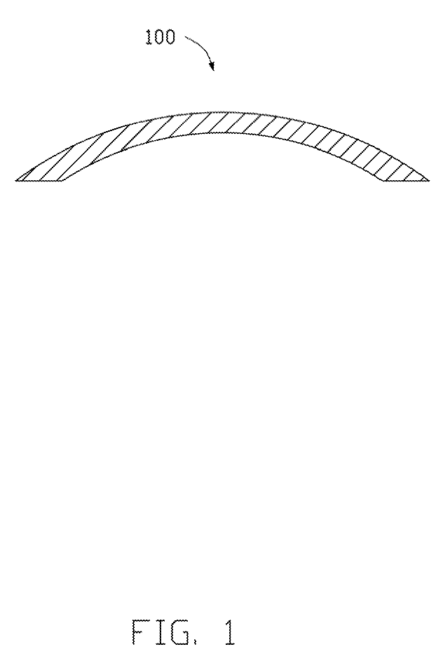

An exemplary embodiment of an artificial cornea stroma material comprises a methacrylated hyaluronic acid, a collagen, a photo-initiator, and a solvent. The artificial cornea stroma material is configured to make an artificial cornea stroma 100 shown in FIG. 1.

The methacrylated hyaluronic acid has a mass percentage of about 0.25% to about 2.5% of a total mass of the artificial cornea stroma material. The collagen has a mass percentage of about 0.005% to about 0.8% of a total mass of the artificial cornea stroma material. The photo-initiator has a mass percentage of about 0.0005% to about 0.05% of a total mass of the artificial cornea stroma material. The solvent has a mass percentage of about 97% to about 99.7% of a total mass of the artificial cornea stroma material.

The photo-initiator absorbs ultraviolet radiation to generate free radicals or positive ions when the artificial cornea stroma material is exposed to ultraviolet radiation. The free radicals or positive ions cause the methacrylated hyaluronic acid to polymerize to form a dense covalent bond cross-linking network gel structure. The cross-linking network gel structure has mechanical strength. The cross-linking network gel structure is stable, thereby the cross-linking network gel structure can keep in gel state for a long time, thereby when an artificial cornea stroma 100 making by the artificial cornea stroma material is implanted in an eye, the artificial cornea stroma 100 will not degrade in a short time.

Changing the mass percentage of the methacrylated hyaluronic acid in the artificial cornea stroma material changes the elastic property of the artificial cornea stroma 100. For example, increasing the mass percentage of the methacrylated hyaluronic acid in the artificial cornea stroma material, the elastic property of the artificial cornea stroma 100 will be better.

The collagen may be selected from commercially available I-type collagen, II-type coppagen, III-type collagen, IV-type collagen, or any combination thereof.

The photo-initiator may be selected from benzoin methyl ether, diethoxyacetophenone, a benzoylphosphine oxide initiator, 1-hydroxycyclohexyl phenyl ketone, Darocure type photo-initiator, Irgacure type photo-initiator, or any combination thereof. In at least one exemplary embodiment, the photo-initiator is selected from Darocure-1173, Darocure-2959, Irgacure-1173, Irgacure-2959, or any combination thereof. The benzoylphosphine oxide initiator may be selected from 2,4,6-trimethylbenzoyldiphenylophosphine oxide, bis-(2,6-dichlorobenzoyl)-4-N-propylphenylphosphine oxide, bis-(2,6-dichlorobenzoyl)-4-N-butylphenylphosphine oxide, or any combination thereof.

The solvent may be acetum. The acetum has a mass concentration of about 0.01 mol/L to about 0.08 mol/L. In at least one exemplary embodiment, the acetum has a mass concentration of 0.05 mol/L.

FIG. 1 illustrates an artificial cornea stroma 100. The artificial cornea stroma 100 is made from the artificial cornea stroma material.

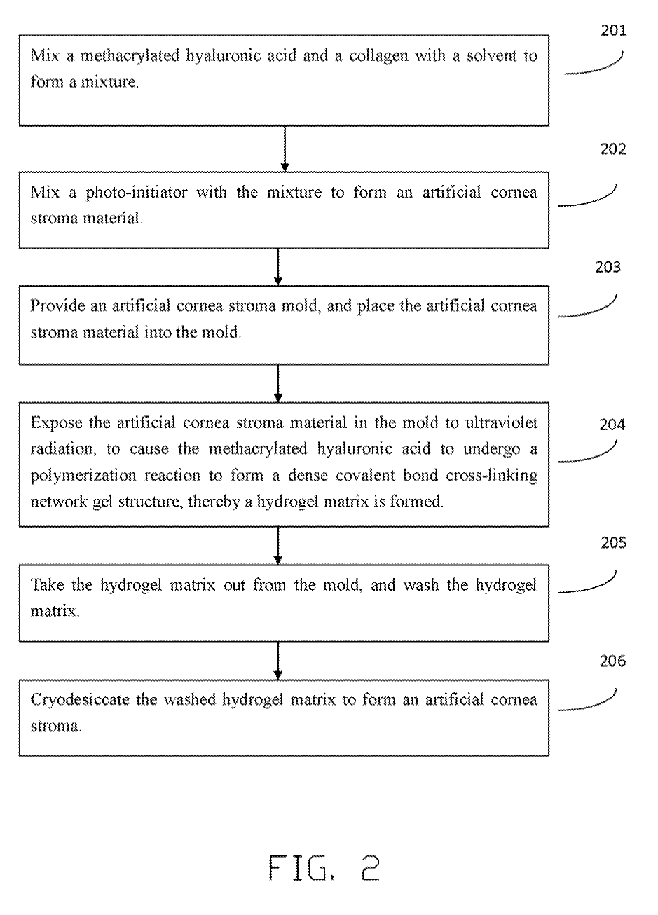

FIG. 2 illustrates a flowchart of a method for manufacturing the artificial cornea stroma 100 in accordance with an exemplary embodiment. The exemplary method is provided by way of example, as there are a variety of ways to carry out the method. Each block shown in FIG. 2 represents one or more processes, methods or subroutines, carried out in the exemplary method. Furthermore, the illustrated order of blocks is by example only, and the order of the blocks can change. Additional blocks may be added, or fewer blocks may be utilized, without departing from this disclosure. The exemplary method may begin at block 201.

At block 201, a methacrylated hyaluronic acid and a collagen are mixed with a solvent to form a mixture.

At block 202, a photo-initiator is mixed with the mixture to form an artificial cornea stroma material. The methacrylated hyaluronic acid has a mass percentage of about 0.25% to about 2.5% of a total mass of the artificial cornea stroma material. The collagen has a mass percentage of about 0.005% to about 0.8% of a total mass of the artificial cornea stroma material. The photo-initiator has a mass percentage of about 0.0005% to about 0.05% of a total mass of the artificial cornea stroma material. The solvent has a mass percentage of about 97% to about 99.7% of a total mass of the artificial cornea stroma material.

At block 203, an artificial cornea stroma mold is provided, and the artificial cornea stroma material is placed into the mold. The artificial cornea stroma mold is transparent.

At block 204, the artificial cornea stroma material in the mold is exposed to ultraviolet radiation, to cause the methacrylated hyaluronic acid to undergo a polymerization reaction to form a dense covalent bond cross-linking network gel structure, thereby a hydrogel matrix is formed.

At block 205, the hydrogel matrix is taken out from the mold, and is washed.

At block 206, the washed hydrogel matrix is cryodesiccated to form an artificial cornea stroma 100.

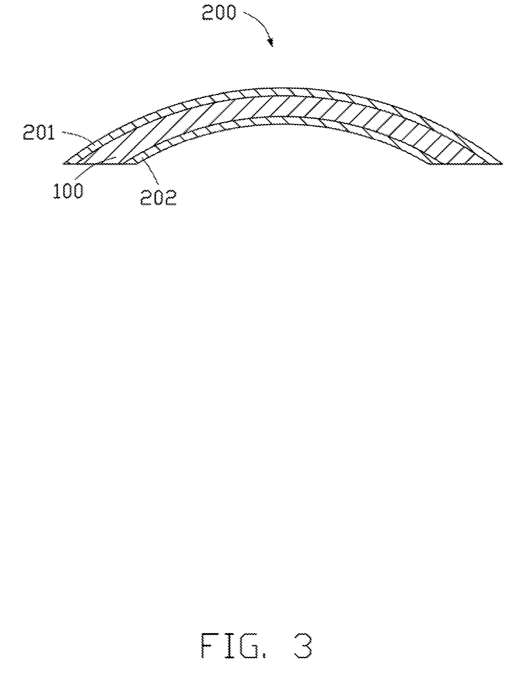

FIG. 3 illustrates an artificial cornea scaffold 200 comprising the artificial cornea stroma 100, an imitation Bowman's layer 201, and an imitation Descemet's membrane 202. The imitation Bowman's layer 201 and the imitation Descemet's membrane 202 are arranged at opposite surfaces of the artificial cornea stroma 100.

When the artificial cornea scaffold 200 is implanted into an eye, the imitation Bowman's layer 201 is at a side of the artificial cornea stroma 100 facing outside of the eye, and the imitation Descemet's membrane 202 is at a side of the artificial cornea stroma 100 facing inside of the eye.

Both the imitation Bowman's layer 201 and the imitation Descemet's membrane 202 comprise collagen. The collagen may be selected from commercially available I-type collagen, II-type coppagen, III-type collagen, IV-type collagen, or any combination thereof.

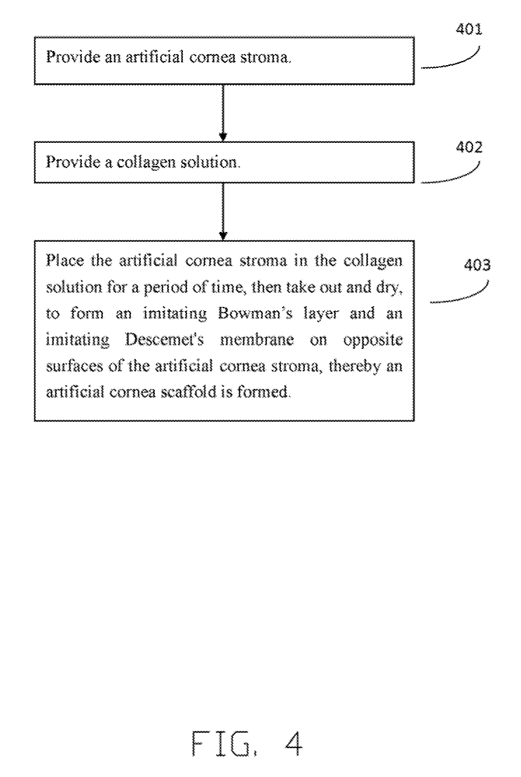

FIG. 4 illustrates a flowchart of a method for manufacturing the artificial cornea scaffold 200 in accordance with an exemplary embodiment. The exemplary method is provided by way of example, as there are a variety of ways to carry out the method. Each block shown in FIG. 4 represents one or more processes, methods or subroutines, carried out in the exemplary method. Furthermore, the illustrated order of blocks is by example only, and the order of the blocks can change. Additional blocks may be added, or fewer blocks may be utilized, without departing from this disclosure. The exemplary method may begin at block 401.

At block 401, an artificial cornea stroma 100 is provided. The artificial cornea stroma 100 is made from an artificial cornea stroma material. The artificial cornea stroma material comprises a methacrylated hyaluronic acid, a collagen, a photo-initiator, and a solvent. The methacrylated hyaluronic acid has a mass percentage of about 0.25% to about 2.5% of a total mass of the artificial cornea stroma material. The collagen has a mass percentage of about 0.005% to about 0.8% of a total mass of the artificial cornea stroma material. The photo-initiator has a mass percentage of about 0.0005% to about 0.05% of a total mass of the artificial cornea stroma material. The solvent has a mass percentage of about 97% to about 99.7% of a total mass of the artificial cornea stroma material.

At block 402, a collagen solution is provided. The collagen solution comprises a collagen. The collagen has a mass percentage of about 0.1% to about 2% of a total mass of the collagen solution. The collagen may be selected from commercially available I-type collagen, II-type coppagen, III-type collagen, IV-type collagen, or any combination thereof.

At block 403, the artificial cornea stroma 100 is placed in the collagen solution for a period of time, then taken out and dried, to form an imitation Bowman's layer 201 and an imitation Descemet's membrane 202 on opposite surfaces of the artificial cornea stroma 100. Thereby an artificial cornea scaffold 200 is formed. In at least one exemplary embodiment, the period of time is about 5 minutes to about 60 minutes.

### EXAMPLE 1

The artificial cornea stroma material comprised methacrylated hyaluronic acid, I-type collagen, Irgacure-1173, and acetum. The acetum had a mass concentration of 0.05 mol/L.

The methacrylated hyaluronic acid had a mass percentage of 0.48% of a total mass of the artificial cornea stroma material. The I-type collagen had a mass percentage of 0.012% of a total mass of the artificial cornea stroma material. The Irgacure-1173 had a mass percentage of 0.034% of a total mass of the artificial cornea stroma material. The acetum had a mass percentage of 99.474% of a total mass of the artificial cornea stroma material.

An artificial cornea stroma made by the artificial cornea stroma material underwent a compression test, the result showed that the artificial cornea stroma can bear a pressure of 86 Pa.

### EXAMPLE 2

The artificial cornea stroma material comprised methacrylated hyaluronic acid, I-type collagen, Irgacure-1173, and acetum. The acetum had a mass concentration of 0.05 mol/L.

The methacrylated hyaluronic acid had a mass percentage of 0.83% of a total mass of the artificial cornea stroma material. The I-type collagen had a mass percentage of 0.012% of a total mass of the artificial cornea stroma material. The Irgacure-1173 had a mass percentage of 0.034% of a total mass of the artificial cornea stroma material. The acetum had a mass percentage of 99.124% of a total mass of the artificial cornea stroma material.

An artificial cornea stroma made by the artificial cornea stroma material underwent a compression test, the result showed that the artificial cornea stroma can bear a pressure of 203 Pa.

### EXAMPLE 3

The artificial cornea stroma material comprised methacrylated hyaluronic acid, I-type collagen, Irgacure-1173, and acetum. The acetum had a mass concentration of 0.05 mol/L.

The methacrylated hyaluronic acid had a mass percentage of 1.02% of a total mass of the artificial cornea stroma material. The I-type collagen had a mass percentage of 0.012% of a total mass of the artificial cornea stroma material. The Irgacure-1173 had a mass percentage of 0.034% of a total mass of the artificial cornea stroma material. The acetum had a mass percentage of 98.934% of a total mass of the artificial cornea stroma material.

An artificial cornea stroma made by the artificial cornea stroma material underwent a compression test, the result showed that the artificial cornea stroma can bear a pressure of 365 Pa.

### EXAMPLE 4

The artificial cornea scaffold is made by the artificial cornea stroma made by the artificial cornea stroma material of example 2.

The artificial cornea stroma was placed in a collagen solution for 30 minutes, then taken out and dried, thereby forming the artificial cornea scaffold. In the collagen solution, the collagen had a mass percentage of 0.48% of a total mass of the collagen solution.

The artificial cornea scaffold underwent a compression test, the result showed that the artificial cornea scaffold can bear a pressure of 198 Pa.

### EXAMPLE 5

The artificial cornea scaffold is made by the artificial cornea stroma made by the artificial cornea stroma material of example 3.

The artificial cornea stroma was placed in a collagen solution for 60 minutes, then taken out and dried, thereby forming the artificial cornea scaffold. In the collagen solution, the collagen had a mass percentage of 0.56% of a total mass of the collagen solution.

The artificial cornea scaffold underwent a compression test, the result showed that the artificial cornea scaffold can bear a pressure of 384 Pa.

It is to be understood, even though information and advantages of the present embodiments have been set forth in the foregoing description, together with details of the structures and functions of the present embodiments, the disclosure is illustrative only; changes may be made in detail, especially in matters of shape, size, and arrangement of parts within the principles of the present embodiments to the full extent indicated by the plain meaning of the terms in which the appended claims are expressed.

What is claimed is:

1. An artificial cornea stroma material comprising:
a methacrylated hyaluronic acid;
a collagen;
a photo-initiator; and
a solvent.

2. The artificial cornea stroma material of claim 1, wherein the methacrylated hyaluronic acid has a mass percentage of about 0.25% to about 2.5% of a total mass of the artificial cornea stroma material, the collagen has a mass percentage of about 0.005% to about 0.8% of a total mass of the artificial cornea stroma material, the photo-initiator has a mass percentage of about 0.0005% to about 0.05% of a total mass of the artificial cornea stroma material, the solvent has a mass percentage of about 97% to about 99.7% of a total mass of the artificial cornea stroma material.

3. The artificial cornea stroma material of claim 1, wherein the collagen is selected from commercially available I-type collagen, II-type coppagen, III-type collagen, IV-type collagen, and any combination thereof.

4. The artificial cornea stroma material of claim 1, wherein the photo-initiator is selected from benzoin methyl ether, diethoxyacetophenone, a benzoylphosphine oxide initiator, 1-hydroxycyclohexyl phenyl ketone, Darocure type photo-initiator, Irgacure type photo-initiator, and any combination thereof.

5. The artificial cornea stroma material of claim 4, wherein the benzoylphosphine oxide initiator is selected from 2,4,6-trimethylbenzoyldiphenylophosphine oxide, bis-(2,6-dichlorobenzoyl)-4-N-propylphenylphosphine oxide, bis-(2,6-dichlorobenzoyl)-4-N-butylphenylphosphine oxide, and any combination thereof.

6. The artificial cornea stroma material of claim 1, wherein the solvent is acetum.

7. The artificial cornea stroma material of claim 6, wherein the acetum has a mass concentration of about 0.01 mol/L to about 0.08 mol/L.

8. An artificial cornea stroma made by:
an artificial cornea stroma material, the artificial cornea stroma material comprising:
a methacrylated hyaluronic acid;
a collagen;
a photo-initiator; and
a solvent.

9. The artificial cornea stroma of claim 8, wherein the methacrylated hyaluronic acid has a mass percentage of about 0.25% to about 2.5% of a total mass of the artificial cornea stroma material, the collagen has a mass percentage of about 0.005% to about 0.8% of a total mass of the artificial cornea stroma material, the photo-initiator has a mass percentage of about 0.0005% to about 0.05% of a total mass of the artificial cornea stroma material, the solvent has a mass percentage of about 97% to about 99.7% of a total mass of the artificial cornea stroma material.

10. The artificial cornea stroma of claim 8, wherein the collagen is selected from commercially available I-type collagen, II-type coppagen, III-type collagen, IV-type collagen, and any combination thereof.

11. The artificial cornea stroma of claim 8, wherein the photo-initiator is selected from benzoin methyl ether, diethoxyacetophenone, a benzoylphosphine oxide initiator, 1-hydroxycyclohexyl phenyl ketone, Darocure type photo-initiator, Irgacure type photo-initiator, and any combination thereof.

12. The artificial cornea stroma of claim 11, wherein the benzoylphosphine oxide initiator is selected from 2,4,6-trimethylbenzoyldiphenylophosphine oxide, bis-(2,6-dichlorobenzoyl)-4-N-propylphenylphosphine oxide, bis-(2,6-dichlorobenzoyl)-4-N-butylphenylphosphine oxide, and any combination thereof.

13. The artificial cornea stroma of claim 8, wherein the solvent is acetum.

14. The artificial cornea stroma of claim 13, wherein the acetum has a mass concentration of about 0.01 mol/L to about 0.08 mol/L.

15. An artificial cornea scaffold comprising:
an imitation Bowman's layer;
an imitation Descemet's membrane; and
an artificial cornea stroma, the imitation Bowman's layer and the imitation Descemet's membrane are arranged at two opposite surfaces of the artificial cornea stroma, the artificial cornea stroma is made by:
an artificial cornea stroma material, the artificial cornea stroma material comprising:
a methacrylated hyaluronic acid;
a collagen;
a photo-initiator; and
a solvent.

16. The artificial cornea scaffold of claim 15, wherein the methacrylated hyaluronic acid has a mass percentage of about 0.25% to about 2.5% of a total mass of the artificial cornea stroma material, the collagen has a mass percentage of about 0.005% to about 0.8% of a total mass of the artificial cornea stroma material, the photo-initiator has a mass percentage of about 0.0005% to about 0.05% of a total mass of the artificial cornea stroma material, the solvent has a mass percentage of about 97% to about 99.7% of a total mass of the artificial cornea stroma material.

17. The artificial cornea scaffold of claim 15, wherein the collagen is selected from commercially available I-type collagen, II-type coppagen, III-type collagen, IV-type collagen, and any combination thereof.

18. The artificial cornea scaffold of claim 15, wherein the photo-initiator is selected from benzoin methyl ether, diethoxyacetophenone, a benzoylphosphine oxide initiator, 1-hydroxycyclohexyl phenyl ketone, Darocure type photo-initiator, Irgacure type photo-initiator, and any combination thereof, the benzoylphosphine oxide initiator is selected from 2,4,6-trimethylbenzoyldiphenylophosphine oxide, bis-(2,6-dichlorobenzoyl)-4-N-propylphenylphosphine oxide, bis-(2,6-dichlorobenzoyl)-4-N-butylphenylphosphine oxide, and any combination thereof.

19. The artificial cornea scaffold of claim 15, wherein the solvent is acetum.

20. The artificial cornea scaffold of claim 19, wherein the acetum has a mass concentration of about 0.01 mol/L to about 0.08 mol/L.
